(19) United States
(12) Patent Application Publication
(10) Pub. No.: US 2009/0215775 A1
(43) Pub. Date: Aug. 27, 2009

(54) Sulphonamido-Substituted Cyclohexyl Sulphones for Treatment of Cancer

(76) Inventors: Huw David Lewis, Sawbridgeworth (GB); Timothy Harrison, Belfast (GB); Mark Steven Shearman, Newton, MA (US)

Correspondence Address: MERCK AND CO., INC, P O BOX 2000, RAHWAY, NJ, 07065-0907, US

(21) Appl. No.: 11/920,451

(22) PCT Filed: May 16, 2006
(86) PCT No.: PCT/GB2006/050109

§ 371 Date: Feb. 17, 2009

(30) Foreign Application Priority Data
May 17, 2005 (GB) 0509932.0
Oct. 24, 2005 (GB) 0521547.0

(51) Int. Cl. A61K 31/5377; A61K 31/18; A61K 31/381; A61K 31/44; A61K 31/425; A61K 31/4196; A61K 31/42; A61K 31/41; A61K 31/4164; A61K 31/415; A61K 31/5375; A61K 31/4427; A61P 35/00

(52) U.S. Cl. 514/235.5; 514/605; 514/445; 514/345; 514/372; 514/384; 514/376; 514/381; 514/398; 514/407; 514/238.2; 514/336

(57) ABSTRACT

Compounds of formula (I) are disclosed for treatment of cancer.

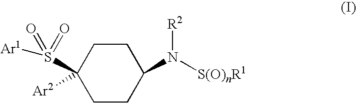

This invention relates to methods and materials for treatment of the human or animal body. In particular it relates to the use of a particular class of sulphones for treatment of cancer.

Notch signalling plays an important part in various cellular and developmental processes, including differentiation, proliferation, survival and apoptosis (Artavaris—Tsakonas et al, Science (1999), 284, 770-776). A significant body of evidence also indicates that augmented or abnormally-prolonged Notch signalling is involved in tumorigenesis (see, for example, Callahan and Egan, J. Mammary Gland Biol. Neoplasia (2004), 9, 145-163; Collins et al, Semin. Cancer Biol. (2004), 14, 357-64; Axelson, ibid. (2004), 14, 317-319; Zweidler-McKay and Pear, ibid (2004), 14, 329-340; and Weng et al, Mol. Cell. Biol. (2003), 23, 655-664).

Modified Notch1 signalling has been implicated in lymphoblastic leukemia/lymphomas, mammary gland tumors, lung cancer, neuroblastomas, skin cancer, cervical cancer, epithelial tumors and prostate cancer. (Allenspach et. al., Cancer Biology and Therapy, (2002) 1:5, 466-476).

Activating mutations in Notch1 are implicated in human T Cell Acute Lymphoblastic Leukemia (T-ALL) (Weng, et al., Science, 306:269-271 (2004)).

Notch signalling is elicited by receptor-ligand interaction between neighbouring cells. As a result of the receptor-ligand interaction, the Notch protein undergoes intra-membrane proteolysis, releasing an intracellular fragment which migrates to the nucleus where it modulates gene expression.

In view of the involvement in tumorigenesis, there has been much interest in inhibition of Notch signalling as a method of treating malignancies. Various types of intervention in the signalling process have been considered, such as inhibiting expression of the Notch protein, blockade of the receptor to prevent ligand binding, and inhibition of the intra-membrane proteolysis. The last-named is particularly attractive because the enzyme complex responsible for the proteolysis, gamma-secretase, has been extensively studied in connection with the cleavage of other protein substrates, notably amyloid precursor protein (APP) which is implicated in Alzheimer's disease. Hence a large number of compounds have been identified which can be shown to inhibit the cleavage of APP by gamma-secretase in vitro. The relevant compounds typically show equivalent ability to inhibit the cleavage of Notch protein by gamma-secretase in vitro (see Lewis et al Biochemistry (2003), 42, 7580-7586). However, clinical studies using such compounds have been severely hampered by the discovery of serious gastro-intestinal (GI) toxicity (believed to be mechanism based) associated with this class of compound (Searfoss et al, J. Bio. Chem. (2003), 278, 46107-46116; Wong et al, ibid (2004), 279, 12876-12882).

It has now been unexpectedly found that a particular class of sulfone derivatives can provide significant inhibition of gamma-secretase in vivo without causing the GI toxicity seen previously with other gamma-secretase inhibitors. This valuable property renders the compounds suitable for use in treating disorders associated with Notch signalling activity, in particular cancer.

Therefore, in accordance with the invention there is provided the use, for the manufacture of a medicament for treating cancer, of a compound of formula I:

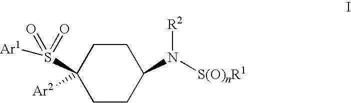

wherein n is 1 or 2;

R¹ represents CF₃ or C₁₋₆alkyl, C₂₋₆alkenyl, C₃₋₉cycloalkyl or C₃₋₆cycloalkylC₁₋₆alkyl, any of which may bear up to 2 substituents selected from halogen, CN, CF₃, OR³, COR³, CO₂R³, OCOR⁴, SO₂R⁴, N(⁵)₂, and CON(⁵)₂,

or R¹ represents aryl, arylC₁₋₆alkyl, C-heterocyclyl or C-heterocyclylC₁₋₆alkyl;

R² represents H or C₁₋₄alkyl;

R³ represents H, C₁₋₄alkyl, phenyl or heteroaryl;

R⁴ represents C₁₋₄alkyl, phenyl or heteroaryl;

R⁵ represents H or C₁₋₄alkyl, or two R⁵ groups together with a nitrogen atom to which they are mutually attached complete an azetidine, pyrrolidine, piperidine, morpholine, thiomorpholine or thiomorpholine-1,1-dioxide ring;

Ar¹ and Ar² independently represent phenyl or heteroaryl, either of which bears 0-3 substituents independently selected from halogen, CN, NO₂, CF₃, CHF₂, OH, OCF₃, CHO, CH═NOH, C₁₋₄alkoxy, C₁₋₄alkoxycarbonyl, C₂₋₆acyl, C₂₋₆alkenyl and C₁₋₄alkyl which optionally bears a substituent selected from halogen, CN, NO₂, CF₃, OH and C₁₋₄alkoxy;

“aryl” at every occurrence thereof refers to phenyl or heteroaryl which optionally bear up to 3 substituents selected from halogen, CN, NO₂, CF₃, OCF₃, OR³, COR³, CO₂R³, OCOR⁴, N(R⁵)₂, CON(R⁵)₂ and optionally-substituted C₁₋₆alkyl, C₁₋₆alkoxy, C₂₋₆alkenyl or C₂₋₆alkenyloxy wherein the substituent is selected from halogen, CN, CF₃, phenyl, OR³, CO₂R³, OCOR⁴, N(R⁵)₂ and CON(R⁵)₂; and

“C-heterocyclyl” and “N-heterocyclyl” at every occurrence thereof refer respectively to a heterocyclic ring system bonded through carbon or nitrogen, said ring system being non-aromatic and comprising up to 10 atoms, at least one of which is O, N or S, and optionally bearing up to 3 substituents selected from oxo, halogen, CN, NO₂, CF₃, OCF₃, OR³, COR³, CO₂R³, OCOR⁴, OSO₂R⁴, N(R⁵)₂, CON(R⁵)₂ and optionally-substituted phenyl, C₁₋₆alkyl, C₁₋₆alkoxy, C₂₋₆alkenyl or C₂₋₆alkenyloxy wherein the substituent is selected from halogen, CN, CF₃, OR³, CO₂R³, OCOR⁴, N(R⁵)₂ and CON(R⁵)₂;

or a pharmaceutically acceptable salt thereof.

According to another aspect of the invention there is provided a method of treating a subject suffering from cancer comprising administering to that subject an effective amount of a compound of formula I as defined above, or a pharmaceutically acceptable salt thereof.

The subject is preferably a mammal, in particular a human.

Where a variable occurs more than once in formula I, the individual occurrences are independent of each other, unless otherwise indicated.

As used herein, the expression “C₁₋ₓalkyl” where x is an integer greater than 1 refers to straight-chained and branched alkyl groups wherein the number of constituent carbon atoms is in the range 1 to x. Particular alkyl groups include methyl, ethyl, n-propyl, isopropyl and t-butyl. Derived expressions such as “C₂₋₆alkenyl”, “hydroxyC₁₋₆alkyl”, “heteroarylC₁₋₆alkyl”, “C₂₋₆alkynyl” and “C₂₋₆alkoxy” are to be construed in an analogous manner.

The expression “C₃₋₉cycloalkyl” as used herein refers to nonaromatic monocyclic or fused bicyclic hydrocarbon ring systems comprising from 3 to 9 ring atoms. Examples include cyclopropyl, cyclobutyl, cyclopentyl, cyclohexyl, cyclohexenyl and bicyclo[2.2.1]heptyl. Monocyclic systems of 3 to 6 members are preferred.

The expression “C₃₋₆ cycloalkylC₁₋₆alkyl” as used herein includes cyclopropylmethyl, cyclobutylmethyl, cyclopentylmethyl and cyclohexylmethyl.

The expression “C₂₋₆acyl” as used herein refers to C₁₋₅alkylcarbonyl groups in which the alkyl portion may be straight chain, branched or cyclic, and may be halogenated. Examples include acetyl, propionyl and trifluoroacetyl.

The expression “heterocyclyl” as defined herein includes both monocyclic and fused bicyclic systems of up to 10 ring atoms selected from C, N, O and S. Mono- or bicyclic systems of up to 7 ring atoms are preferred, and monocyclic systems of 4, 5 or 6 ring atoms are most preferred. Examples of heterocyclic ring systems include azetidinyl, pyrrolidinyl, 3-pyrrolinyl, terahydrofuryl, 1,3-dioxolanyl, tetrahydrothiophenyl, tetrahydropyridinyl, piperidinyl, piperazinyl, morpholinyl, thiomorpholinyl, imidazolidinyl, oxazolidinyl, thiazolidinyl, 2,5-diazabicyclo[2.2.1]heptyl, 2-aza-5-oxabicyclo[2.2.1]heptyl and 1,4-dioxa-8-azaspiro[4.5]decanyl. Unless otherwise indicated, heterocyclyl groups may be bonded through a ring carbon atom or a ring nitrogen atom where present. “C-heterocyclyl” indicates bonding through carbon, while “N-heterocyclyl” indicates bonding through nitrogen.

The expression “heteroaryl” as used herein means a monocyclic system of 5 or 6 ring atoms, or fused bicyclic system of up to 10 ring atoms, selected from C, N, O and S, wherein at least one of the constituent rings is aromatic and comprises at least one ring atom which is other than carbon. Monocyclic systems of 5 or 6 members are preferred. Examples of heteroaryl groups include pyridinyl, pyridazinyl, pyrimidinyl, pyrazinyl, pyrrolyl, furyl, thienyl, pyrazolyl, oxazolyl, isoxazolyl, thiazolyl, isothiazolyl, imidazolyl, oxadiazolyl, triazolyl and thiadiazolyl groups and benzo-fused analogues thereof. Further examples of heteroaryl groups include tetrazole, 1,2,4-triazine and 1,3,5-triazine. Pyridine rings may be in the N-oxide form.

Where a phenyl group or heteroaryl group bears more than one substituent, preferably not more than one of said substituents is other than halogen or alkyl. Where an alkyl group bears more than one substituent, preferably not more than one of said substituents is other than halogen.

The term “halogen” as used herein includes fluorine, chlorine, bromine and iodine, of which fluorine and chlorine are preferred.

For use in medicine, the compounds of formula I may advantageously be in the form of pharmaceutically acceptable salts. Other salts may, however, be useful in the preparation of the compounds of formula I or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, benzenesulfonic acid, methanesulfonic acid, fumaric acid, maleic acid, succinic acid, acetic acid, benzoic acid, oxalic acid, citric acid, tartaric acid, carbonic acid or phosphoric acid. Alternatively, where the compound of the invention carries an acidic moiety, a pharmaceutically acceptable salt may be formed by neutralisation of said acidic moiety with a suitable base. Examples of pharmaceutically acceptable salts thus formed include alkali metal salts such as sodium or potassium salts; ammonium salts; alkaline earth metal salts such as calcium or magnesium salts; and salts formed with suitable organic bases, such as amine salts (including pyridinium salts) and quaternary ammonium salts.

Where the compounds according to formula I have at least one asymmetric centre, they may accordingly exist as enantiomers. Where the compounds according to the invention possess two or more asymmetric centres, they may additionally exist as diastereoisomers. It is to be understood that all such isomers and mixtures thereof in any proportion are encompassed within the scope of the present invention.

In the compounds of formula I, n is 1 or 2, preferably 2.

R¹ is preferably CF₃, aryl or arylalkyl, or an alkyl, alkenyl, cycloalkyl or cycloalkylalkyl group, optionally substituted as described previously. Preferred substituents include halogen (especially fluorine or chlorine), CF₃, CN, OR³ (especially OH, OMe and OEt), COR³ (especially acetyl), CO₂R³ (especially CO₂H, CO₂Me and CO₂Et), SO₂R⁴ (especially methanesulfonyl), N(R⁵)₂ (especially when the R⁵ groups complete a ring) and CON(R⁵)₂ (especially CONH₂).

Examples of alkyl groups represented by R¹ include methyl, ethyl, n-propyl, isopropyl, n-butyl, t-butyl, isobutyl, 2,2,2-trifluoroethyl, chloromethyl, 3-chloropropyl, 2-chloro-2-propyl, cyanomethyl, 2-hydroxyethyl, 2-methoxyethyl, 2-hydroxy-2-methylpropyl, carboxymethyl, methoxycarbonylmethyl, 1-carboxyethyl, 1-ethoxycarbonylethyl, carbamoylmethyl, 2-(pyrrolidin-1-yl)ethyl, 2-(morpholin-4-yl)ethyl and MeSO₂CH₂—.

Examples of alkenyl groups represented by R¹ include vinyl and allyl.

Examples of cycloalkyl and cycloalkylalkyl groups represented by R¹ include cyclopropyl, cyclobutyl, cyclopentyl, cyclohexyl, cyclopropylmethyl and cyclopentylmethyl.

When R¹ represents aryl or arylalkyl, the aryl group may be phenyl or heteroaryl (especially 5- or 6-membered heteroaryl), optionally substituted as defined previously. Preferred substituents include halogen (especially chlorine, bromine or fluorine), CN, CF₃, OCF₃, alkyl (especially methyl), OH, alkoxy (especially methoxy) and alkoxycarbonyl (such as methoxycarbonyl). Preferred heteroaryl groups include pyridine, pyrimidine, furan, thiophene, thiazole, isothiazole, isoxazole, pyrazole, imidazole, triazole, thiadiazole and tetrazole, especially pyridine, furan, thiophene, thiazole, isothiazole, isoxazole, pyrazole, imidazole, triazole, and tetrazole.

Examples of aryl groups represented by R¹ include phenyl, 2-, 3- and 4-fluorophenyl, 2,4-difluorophenyl, 2-chlorophenyl, 2-bromophenyl, 2-cyanophenyl, 2-methylphenyl, 2-trifluoromethylphenyl, 2-methoxyphenyl, 5-chloro-2-methoxyphenyl, 2-pyridyl, 4-pyridyl, 6-chloro-3-pyridyl, 2-furyl, 2-thienyl, 3-thienyl, 2-thiazolyl, 5-isothiazolyl, 2-imidazolyl, 2-methylfuran-3-yl, 5-chloro-2-thienyl, 4-chloro-2-thienyl, 3-chloro-2-thienyl, 3-bromo-2-thienyl, 4-bromo-2-thienyl, 5-methyl-2-thienyl, 2-(methoxycarbonyl)-3-thienyl, 4-methylthiazol-3-yl, 1-methylimidazol-2-yl, 1-methylimidazol-5-yl, 1-methylimidazol-4-yl, 3-chloro-1,5-dimethylpyrazol-4-yl, 3,5-dimethylisoxazol-4-yl, 1-methyl-1,2,3,4-tetrazol-5-yl, 1,2,4-triazol-3-yl, 1-methyl-1,2,4-triazol-3-yl, 2-methyl-1,2,4-triazol-3-yl and 4-methyl-1,2,4-triazol-3-yl.

Arylalkyl groups represented by R¹ are typically optionally substituted benzyl, phenethyl, heteroarylmethyl or heteroarylethyl groups. Examples include benzyl, 2-furylmethyl, 2-thienylmethyl and 1-(2-thienyl)ethyl. Preferred examples include benzyl.

R² preferably represents H or methyl, most preferably H.

R³ preferably represents H, C₁₋₄alkyl, phenyl, pyridyl, or 5-membered heteroaryl. Most preferably, R³ represents H or C₁₋₄alkyl.

R⁴ preferably represents C₁₋₄alkyl, phenyl, pyridyl, or 5-membered heteroaryl. Most preferably, R³ represents C₁₋₄alkyl.

Ar¹ and Ar² independently represent optionally substituted phenyl or heteroaryl. Ar¹ is preferably selected from optionally substituted phenyl and optionally substituted 6-membered heteroaryl. Preferred 6-membered heteroaryl embodiments of Ar¹ include optionally substituted pyridyl, in particular optionally substituted 3-pyridyl. Ar¹ is preferably selected from 6-(trifluoromethyl)-3-pyridyl and phenyl which is optionally substituted in the 4-position with halogen, CN, vinyl, allyl, acetyl, methyl or mono-, di- or trifluoromethyl. In one preferred embodiment of the invention Ar¹ represents 4-chlorophenyl. In another preferred embodiment Ar¹ represents 4-trifluoromethylphenyl. In a further preferred embodiment, Ar¹ represents 6-(trifluoromethyl)-3-pyridyl.

Ar² preferably represents optionally substituted phenyl, in particular phenyl bearing 2 or 3 substituents selected from halogen, CN, CF₃ and optionally-substituted alkyl. Ar² is typically selected from phenyl groups bearing halogen substituents (preferably fluorine) in the 2- and 5-positions or in the 2-, 3- and 6-positions, or from phenyl groups bearing a fluorine substituent in the 2-position and halogen, CN, methyl or hydroxymethyl in the 5-position. In a preferred embodiment of the invention, Ar² represents 2,5-difluorophenyl.

In a particular embodiment, Ar¹ is 6-trifluoromethyl-3-pyridyl, 4-chlorophenyl or 4-trifluoromethylphenyl and Ar² is 2,5-difluorophenyl.

A preferred subclass of the compounds useful in the invention are the compounds of formula II:

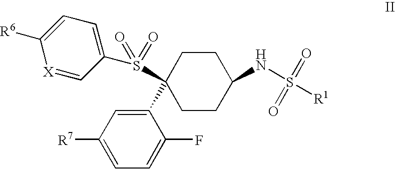

wherein X represents N or CH;

R⁶ represents H, F, Cl, Br, CN, CF₃, CH=CH₂ or CH₃;

R⁷ represents F, Cl, Br, CN, CH₃ or CH₂OH; and

R¹ has the same definition and preferred identities as before;

and pharmaceutically acceptable salts thereof.

When X represents N, R⁶ is preferably CF₃.

In a preferred embodiment, R¹ is selected from:

(a) CF₃;

(b) C₁₋₆alkyl which optionally bears up to 2 substituents selected from halogen, CN, CF₃, OR³, CO₂R³, SO₂R⁴, N(R)₂, and CON(R⁵)₂; and

(c) phenyl, pyridyl or 5-membered heteroaryl which optionally bear up to 3 substituents selected from halogen, CN, CF₃, OR³, COR³, CO₂R³, OCOR⁴, N(R⁵)_{2,} CON(R⁵)₂ and optionally-substituted C₁₋₆alkyl, C₁₋₆alkoxy, C₂₋₆alkenyl or C₂₋₆alkenyloxy wherein the substituent is selected from halogen, CN, CF₃, phenyl, OR³, CO₂R³, OCOR⁴, N(R)₂ and CON(⁵)₂;

where R³, R⁴ and R⁵ have the same definitions and preferred identities as before.

R¹ very aptly represents CF₃.

The compounds of formula I may be prepared as described in WO 2004/031139.

Examples of individual compounds useful in the invention are provided in the Examples section appended hereto.

Cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colon, colorectal, rectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

Cancers that may be treated by the compounds, compositions and methods of the invention include, but are not limited to: breast, prostate, colon, lung, brain, testicular, stomach, pancreas, skin, small intestine, large intestine, throat, head and neck, oral, bone, liver, bladder, kidney, thyroid and blood.

Cancers that may be treated by the compounds, compositions and methods of the invention in particular include all types in which Notch signalling is known to play a role in the initial formation, proliferation or metastasis of cancerous cells. Modified Notch1 signalling has been implicated in lymphoblastic leukemia/lymphomas, mammary gland tumors, lung cancer, neuroblastomas, skin cancer, cervical cancer, epithelial tumors and prostate cancer. (Allenspach et. al., Cancer Biology and Therapy, 1:5, 466-476, 2002). Therefore compounds of the instant invention are useful in the treatment of the above described cancers.

Activating mutations in Notch1 are implicated in human T Cell Acute Lymphoblastic Leukemia (T-ALL) (Weng, et al., Science, 306:269-271 (2004)). Compounds of the instant invention are therefore useful in the treatment of T-ALL.

Cancers that may be treated by the compounds, compositions and methods of the invention include: breast, prostate, colon, ovarian, colorectal, brain and lung.

Cancers that may be treated by the compounds, compositions and methods of the invention include: lymphoma and leukemia.

Cancers that may be treated by the compounds, compositions and methods of the invention include breast cancer.

Cancers that may be treated by the compounds, compositions and methods of the invention include lung cancer, in particular non-small cell lung cancer.

Cancers that may be treated by the compounds, compositions and methods of the invention include colon cancer and colorectal cancer.

Cancers that may be treated by the compounds, compositions and methods of the invention include brain cancer, including glioma, medulloblastoma and ependymoma.

Cancers that may be treated by the compounds, compositions and methods of the invention include familial adenomatous polyposis (FAP).

Cancers that may be treated by the compounds, compositions and methods of the invention include Barrett's esophagus.

Exposure to compounds of the instant invention has been shown to cause cell cycle arrest, in particular G₀/G₁ arrest, in populations of cells with a high level of Notch expression, but not in populations lacking such expression. Furthermore, it has been found that the arrested cells selectively undergo apoptosis. Hence, the compounds of the instant invention have the potential to selectively target malignant cells without damaging neighbouring healthy cells.

The compounds of the instant invention are suitable for treating cancer via the selective targeting of cancer stem cells.

The compounds of formula I may be administered to mammals, including humans, either alone or, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid parrafin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the instant invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds for the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a composition according to this invention is administered into a human subject, the daily dosage will normally be determined by the prescribing physician with the dosage generally varying according to the age, weight, and response of the individual patient, as well as the severity of the patient's symptoms.

The dosage regimen utilizing the compounds of the instant invention can be selected in accordance with a variety of factors including type, species, age, weight, sex and the type of cancer being treated; the severity (i.e., stage) of the cancer to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the disease. For example, compounds of the instant invention can be administered in a total daily dose of up to 1000 mg. Compounds of the instant invention can be administered once daily (QD), or divided into multiple daily doses such as twice daily (BID), and three times daily (TID). Compounds of the instant invention can be administered at a total daily dosage of up to 1000 mg, e.g., 200 mg, 300 mg, 400 mg, 600 mg, 800 mg or 1000 mg, which can be administered in one daily dose or can be divided into multiple daily doses as described above.

In addition, the administration can be continuous, i.e., every day, or intermittently. The terms “intermittent” or “intermittently” as used herein means stopping and starting at either regular or irregular intervals. For example, intermittent administration of a compound of the instant invention may be administration one to six days per week or it may mean administration in cycles (e.g. daily administration for two to eight consecutive weeks, then a rest period with no administration for up to one week) or it may mean administration on alternate days.

In addition, the compounds of the instant invention may be administered according to any of the schedules described above, consecutively for a few weeks, followed by a rest period. For example, the compounds of the instant invention may be administered according to any one of the schedules described above from two to eight weeks, followed by a rest period of one week, or twice daily at a dose of 100-500 mg for three to five days a week. In another particular embodiment, the compounds of the instant invention may be administered three times daily for two consecutive weeks, followed by one week of rest.

In a further example of intermittent dosing, the compounds of the instant invention are administered on three consecutive days followed by four days of rest.

In a yet further example of intermittent dosing, the compounds of the instant invention are administered on one day, followed by six days of rest.

In a yet further example of intermittent dosing, the compounds of the instant invention are administered on one day, followed by 10 to 13 days of rest.

The instant compounds are also useful in combination with known therapeutic agents and anti-cancer agents. For example, instant compounds are useful in combination with known anti-cancer agents. Combinations of the presently disclosed compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, and agents that interfere with cell cycle checkpoints. The instant compounds are particularly useful when co-administered with radiation therapy.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, 4-demethoxy-3-deamino-3-aziridinyl-4-methylsulphonyl-daunorubicin (see WO 00/50032), Raf kinase inhibitors (such as Bay43-9006) and mTOR inhibitors (such as Wyeth's CCI-779).

An example of a hypoxia activatable compound is tirapazamine.

Examples of proteosome inhibitors include but are not limited to lactacystin and MLN-341 (Velcade).

Examples of microtubule inhibitors/microtubule-stabilising agents include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797. In an embodiment the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H, 12H-benzo[de]pyrano[3′,4′:b,7]-indolizino[1,2b]quinoline-10,13(9H, 15H)dione, lurtotecan, 7-[2-(N-isopropylamino)ethyl]-(20S)camptothecin, BNP1350, BNP1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB,8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in Publications WO03/039460, WO03/050064, WO03/050122, WO03/049527, WO03/049679, WO03/049678, WO04/039774, WO03/079973, WO03/09921 1, WO03/105855, WO03/106417, WO04/037171, WO04/058148, WO04/058700, WO04/126699, WO05/018638, WO05/019206, WO05/019205, WO05/018547, WO05/017190, US2005/0176776. In an embodiment inhibitors of mitotic kinesins include, but are not limited to inhibitors of KSP, inhibitors of MKLP1, inhibitors of CENP-E, inhibitors of MCAK and inhibitors of Rab6-KIFL.

Examples of “histone deacetylase inhibitors” include, but are not limited to, SAHA, TSA, oxamflatin, PXD101, MG98 and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E), 4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p.573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in WO 03/13526.

“Agents that interfere with cell cycle checkpoints” refer to compounds that inhibit protein kinases that transduce cell cycle checkpoint signals, thereby sensitizing the cancer cell to DNA damaging agents.

Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen and Hunter, Nature, 411:355-365, 2001.

“Inhibitors of cell proliferation and survival signalling pathway” refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, 60/734188, 60/652737, 60/670469), inhibitors of Raf kinase (for example BAY-43-9006 ), inhibitors of MEK (for example CI-1040 and PD-098059), inhibitors of mTOR (for example Wyeth CCI-779), and inhibitors of P13K (for example LY294002).

As described above, the combinations with NSAID's are directed to the use of NSAID's which are potent COX-2 inhibiting agents. For purposes of this specification an NSAID is potent if it possesses an IC₅₀ for the inhibition of COX-2 of 1 μM or less as measured by cell or microsomal assays.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272 and U.S. Pat. No. 5,932,598, all of which are hereby incorporated by reference.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to, the following: parecoxib, BEXTRA® and CELEBREX® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide,CM101, squalamine, combretastatin, RP14610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ3 integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′kl]pyrrolo[3,4-i][1,6]benzodiazocin-1-one, SH268, genistein, ST1571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, ST1571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999;274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid WO 01/60807, and 2(R)-7-(3-(2-chloro-4-(4-fluorophenoxy)phenoxy)propoxy)-2-ethylchromane-2-carboxylic acid WO 02/026729.

Another embodiment of the instant invention is the use of the presently disclosed compounds in combination with gene therapy for the treatment of cancer. For an overview of genetic strategies to treating cancer see Hall et al (Am. J Hum. Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998;5(8): 1105-13), and interferon gamma (J. Immunol. 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT₃ receptor antagonists, such as ondansetron, granisetron, tropisetron, and zatisetron, GABA_{B} receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In another embodiment, conjunctive therapy with an anti-emesis agent selected from a neurokinin-1 receptor antagonist, a 5HT₃ receptor antagonist and a corticosteroid is disclosed for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 05 129 02, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913,0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin.

A compound of the instant invention may also be useful for treating or preventing cancer in combination with P450 inhibitors including: xenobiotics, quinidine, tyramine, ketoconazole, testosterone, quinine, methyrapone, caffeine, phenelzine, doxorubicin, troleandomycin, cyclobenzaprine, erythromycin, cocaine, furafyline, cimetidine, dextromethorphan, ritonavir, indinavir, amprenavir, diltiazem, terfenadine, verapamil, cortisol, itraconazole, mibefradil, nefazodone and nelfinavir.

A compound of the instant invention may also be useful for treating or preventing cancer in combination with Pgp and/or BCRP inhibitors including: cyclosporin A, PSC833, GF120918, cremophorEL, fumitremorgin C, Ko132, Ko134, Iressa, Imatnib mesylate, EKI-785, C11033, novobiocin, diethylstilbestrol, tamoxifen, resperpine, VX-710, tryprostatin A, flavonoids, ritonavir, saquinavir, nelfinavir, omeprazole, quinidine, verapamil, terfenadine, ketoconazole, nifidepine, FK506, amiodarone, XR9576, indinavir, amprenavir, cortisol, testosterone, LY335979, OC144-093, erythromycin, vincristine, digoxin and talinolol.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be useful for treating or preventing breast cancer in combination with aromatase inhibitors. Examples of aromatase inhibitors include but are not limited to: anastrozole, letrozole and exemestane.

A compound of the instant invention may also be useful for treating or preventing cancer in combination with siRNA therapeutics.

The compounds of the instant invention may also be administered in combination with other γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139).

A compound of the instant invention may also be useful for treating or preventing cancer in combination with PARP inhibitors.

A compound of the instant invention may also be useful for treating cancer in combination with one or more of the following therapeutic agents: abarelix (Plenaxis depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (dromostanolone®); dromostanolone propionate (masterone injection®); Elliott's B Solution (Elliott's B Solution®); epirubicin (Ellence®); Epoetin alfa (epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); and zoledronate (Zometa®).

Thus, the scope of the instant invention encompasses the use of the instantly claimed compounds in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HW protease inhibitor, a reverse transcriptase inhibitor, an angiogenesis inhibitor, a PPAR-y agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, a γ-secretase and/or NOTCH inhibitor, an agent that interferes with receptor tyrosine kinases (RTKs), an agent that interferes with a cell cycle checkpoint, and any of the therapeutic agents listed above.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering a therapeutically effective amount of a compound of the instant invention in combination with radiation therapy and/or in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, a γ-secretase and/or NOTCH inhibitor, an agent that interferes with receptor tyrosine kinases (RTKs), an agent that interferes with a cell cycle checkpoint, and any of the therapeutic agents listed above.

Yet another embodiment of the invention is a method of treating cancer that comprises administering to a patient in need thereof a therapeutically effective amount of a compound of formula I or a pharmaceutically acceptable salt thereof in combination with a second medicament selected from: paclitaxel (Taxol®, optionally in combination with carboplatin); docetaxel (Taxotere®); trastuzumab (Herceptin®); tamoxifen (Nolvadex®); bevacuzimab (Avastin®); and erlotinib (Tarceva®).

The invention further encompasses a method of treating or preventing cancer that comprises administering to a patient in need thereof a therapeutically effective amount of a compound of formula I or a pharmaceutically acceptable salt thereof in combination with a COX-2 inhibitor.

The instant invention also includes a pharmaceutical composition useful for treating or preventing cancer that comprises a therapeutically effective amount of a compound of the instant invention and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, a γ-secretase and/or NOTCH inhibitor, an agent that interfere with receptor tyrosine kinases (RTKs), an agent that interferes with a cell cycle checkpoint, and any of the therapeutic agents listed above.

Any of the specific dosages and dosage schedules applicable to the compounds of the instant invention may also be applicable to the therapeutic agents to be used in a combination treatment (hereinafter referred to as the “second therapeutic agent”).

Moreover, the specific dosage and dosage schedule of this second therapeutic agent can further vary, and the optimal dose, dosing schedule and route of administration will be determined based upon the specific second therapeutic agent that is being used.

Of course, the route of administration of the compounds of the instant invention is independent of the route of administration of the second therapeutic agent. In an embodiment, the administration for a compound of the instant invention is oral administration. In another embodiment, the administration for a compound of the instant invention is intravenous administration. Thus, in accordance with these embodiments, a compound of the instant invention is administered orally or intravenously, and the second therapeutic agent can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

In addition, a compound of the instant invention and second therapeutic agent may be administered by the same mode of administration, i.e. both agents administered e.g. orally or intravenously. However, it is also within the scope of the present invention to administer a compound of the instant invention by one mode of administration, e.g. orally, and to administer the second therapeutic agent by another mode of administration, e.g. intravenously or by any of the other administration modes described hereinabove.

The first treatment procedure, administration of a compound of the instant invention, can take place prior to the second treatment procedure, i.e., the second therapeutic agent, after the treatment with the second therapeutic agent, at the same time as the treatment with the second therapeutic agent, or a combination thereof. For example, a total treatment period can be decided for a compound of the instant invention. The second therapeutic agent can be administered prior to onset of treatment with a compound of the instant invention or following treatment with a compound of the instant invention. In addition, anti-cancer treatment can be administered during the period of administration of a compound of the instant invention but does not need to occur over the entire treatment period of a compound of the instant invention.

The term “administration” and variants thereof (e.g., “administering”) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating cancer” and “treatment of cancer” encompass prophylactic treatments as well as treatments targeting an existing cancerous condition. Thus, the compounds of the instant invention may be administered to a patient alone or in combination with one or more conventional chemotherapeutic, radiotherapeutic or surgical interventions, for the purpose of arresting or attenuating an existing malignant condition by killing cancerous cells. However, said compounds may also be administered simultaneously with or subsequent to a conventional chemotherapeutic, radiotherapeutic or surgical intervention for the purpose of preventing or delaying the recurrence or metastasis of cancerous cells.

All patents, publications and pending patent applications identified are hereby incorporated by reference.

Suitable methods of assaying the level of activity of compounds of the present invention towards γ-secretase are disclosed in WO 01/70677, WO 03/093252, and in Biochemistry, 2000, 39(30), 8698-8704 (APP as substrate); and in Biochemistry (2003), 42, 7580-7586 (Notch as substrate).

The Examples of the present invention all had an ED₅₀ of less than 0.5 μM, in most cases less than 100 nM, and in preferred cases less than 10 nM, in at least one of the above assays.

The following examples illustrate the present invention.

### Assay for Cell Cycle Arrest

Cells expressing Notch (ALL-SIL, DND-41, HPB-ALL or TALL-1) (Weng et al, Science, 306 (2004), 269-71) are incubated in the presence or absence of a compound of the instant invention (e.g. the compound of Example 47 below) at concentrations up to 10 μM. At the end of the incubation (typically 4-8 days), the cells are collected, fixed in 70% ethanol on ice for >2 hours, washed, then labelled for 15 min at 37° C. with propidium iodide (0.2 mg/ml) (PI) in the presence of 0.1% Triton X100 and 0.2 mg/ml RNase and subjected to FACS analysis. In comparison to untreated controls, treated cell cultures show severe loss of G₂- and S-phase populations, consistent with G₀/G₁ arrest.

### Assay for Apoptosis

This assay relies on the detection of phosphatidylserine (PS) on the external surface of apoptotic cells via binding to Annexin V, since PS in intact cells remains inaccessible. The bound Annexin V is labelled with FITC-conjugated antibody for analysis by FACS. Kits for carrying out this assay are available commercially (e.g. from BD cat. no. 556547).

Cells are incubated as described above in the presence of Annexin V, then collected, washed, labelled with FITC-Annexin V and PI antibodies, and analysed by FACS. Due to exposure of PS-bound Annexin V on the outside of apoptotic, but not normal, cells, FACS technologies enables quantification of the population of apoptotic cells highly stained by FITC-conjugated antibodies raised against Annexin V.

Typically, cells treated with 0.1% DMSO for 7 days show negligible accessible expression of Annexin V, the bulk of the cell population remaining unstained. In contrast, exposure to compounds of the instant invention at 10 μM for 7 days (replenished twice during the experiment) leads to a reduction of the number of such cells with low accessibility of Annexin V, and the appearance of a highly labelled population of cells, consistent with known redistribution of this protein during apoptosis. Equivalent apoptosis is not shown when the duration of treatment (4 days) is insufficient to cause cell cycle arrest, or the concentration of inhibitor is insufficient to cause arrest, or when a Notch-independent cell line is used. Furthermore, a titration comparison of representative inhibitors in HPB-ALL cells over 6 days reveals a perfect correlation between the treatments that cause apoptosis and those that cause parallel cell cycle arrest.

### Assay for Cell Viability

Cell lines such as ALL-SIL, DND-41, HPB-ALL, and T-ALL-1 cell lines are seeded to 96 well plates (1×10⁴ cells in 90 μl/well) in media specified by the cell line supplier (DSMZ, German National Resource Centre for Biological Material). Following overnight incubation of 90 μl at 37° C. in 5% CO₂, 10 μl of media containing 10× γ-secretase inhibitor stock is added, yielding a final concentration of 0.1% DMSO. Media containing inhibitor (75 μl) is replaced after a brief centrifugation every 2 days and the cells are completely resuspended. Cell viability is measured following 8 days of treatment using ATPlite (PerkinElmer), according to the manufacturer's instructions.

### Assay to Measure Gamma-Secretase Inhibition by Monitoring Cleavage of the Substrate Notch 1

Treated cells are lysed in buffer containing 1% Triton X-100, 0.5% NP-40, 0.2% SDS in TBS and vortexed. Samples are rocked for 25 minutes at 4° C., sonicated for 15 seconds and centrifuged at 14,000×g to collect supernatant. Protein is quantitated using the Biorad DC Protein assay (#500-0116) and 30-50 μg of protein separated on 10-20% Tricine gel. Proteins are transferred to nitrocellulose membranes, blocked in 10% Milk for 1 hour, and probed with cleaved Notch 1 antibody (#2421, Cell Signaling Technologies) diluted 1:1000 in PBS overnight at 4° C. Membranes washed in PBS are subsequently probed with anti-rabbit-HRP at 1:7000 for 1 h and proteins revealed to film using Pierce SuperSignal West Femto.

### Assay to Measure Inhibition of the Notch Pathway by Monitoring Notch Target Genes Response in Cells or Tumors

RNA is extracted according to the RNeasy kit from Qiagen and cDNA prepared as described by Applied Biosystems using the High Capacity cDNA Archive kit. Notch pathway response genes such as Hes1 and Hes5 are quantitated using Taqman Real-Time PCR with probes purchased from Applied Biosystems.

### Assay for Anti-Tumor Activity

CD1 nude mice predosed with cyclophosphamide (100 mg/kg, i.p. for 3 days) are injected subcutaneously with 5×10⁶ T-ALL-1 cells per mouse in PBS/matrigel. Tumor volume is monitored with calipers and when this reaches ˜250mm³ the mice are dosed orally 4 days-On, 4-days-Off for a period of 24-32 days using inhibitor formulated in 0.5% methylcellulose. Body weight and tumor volume are recorded daily and all procedures are conducted according to IACUC guidelines.

### EXAMPLES

### Intermediate A

### 4-(4-chlorobenzenesulfonyl)-4-(2,5-difluorophenyl)cyclohexylamine

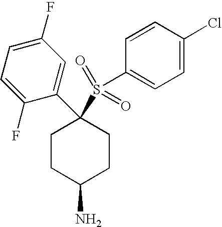

(1) 4-[(4-Chlorophenyl)sulfonyl]-4-(2,5-difluorophenyl)cyclohexanone was prepared as described in WO 02/081435 (Example 2).
This cyclohexanone (0.1 g, 0.26 mmol) in methanol (2 ml) was treated with NaBH₄ (0.098 g, 0.26 mmol), stirred for 1 hour, quenched with HCl (1N, 10 ml), diluted with ethyl acetate (20 ml), then the organic phase was separated, dried (MgSO₄) and evaporated to dryness. The trans 4-[(4-chlorophenyl)sulfonyl]-4-(2,5-difluorophenyl)cyclohexanol was purified on silica eluting with hexane-ethyl acetate mixtures. 0.052 g. ¹H NMR CDCl₃ 7.39-7.33 (4H, m), 7.11-7.02 (2H, m), 6.88-6.82 (1H, m), 3.80-3.73 (1H, m), 2.80-2.60 (2H, m), 2.22-2.16 (2H, m), 2.08-2.04 (2H, m), 1.53(1H, br) and 1.27-1.13 (2H, m).
(2) To this alcohol (2.7 g, 6.9 mmol) and triethylamine (1.45 ml, 10.3 mmol) in dichloromethane (50 ml) was added methanesulfonyl chloride (0.645 ml, 8.9 mmol) at −30° C. After 30 minutes the mixture was washed with water (20 ml), 10% aqueous citric acid (20 ml) and saturated aqueous sodium hydrogen carbonate (50 ml), dried (MgSO₄) and evaporated to dryness. The solid was triturated with ether to give the mesylate (2.6 g).
(3) The mesylate (1.5 g, 3.2 mmol) in dimethylformamide (5 ml) was treated with sodium azide (315 mg, 4.8 mmol) and heated to 90° C. for 6 hrs. The mixture was treated with water (80 ml), and extracted with diethyl ether (3×50 ml), dried (MgSO₄) and evaporated to dryness. The solid was triturated with ether to give the cis azide (1.4 g)
(4) The azide (1 g, 2.55 mmol) in tetrahydrofuran (10 ml) and water (1 ml), was treated with triphenylphosphine (740 mg, 2.8 mmol) at room temperature for 15 mins, water (5 ml) was added and the mixture was heated at reflux for 4 hrs. After cooling to room temperature and passage through SCX Varian Bond Elut™ cartridge, the basic fraction was evaporated to give the primary amine. MS MH+386(388).

### Intermediate B

### 4-(2,5-difluorophenyl)-4-(4-trifluoromethylbenzenesulfonyl)cyclohexylamine

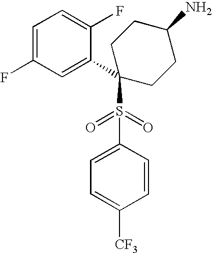

Prepared as for Intermediate A, using the appropriate cyclohexanone (WO 02/081435, Example 41) in step (1), except that the borohydride reduction was carried out at −20° C. MS (ES+) MH+420

### Intermediate C

### 4-(2,5-difluorophenyl)-4-(6-trifluoromethyl-pyridine-3-sulfonyl)-cyclohexylamine

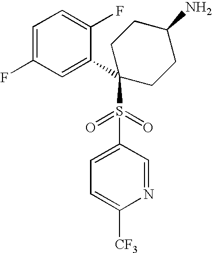

(1) A solution of 3-amino-6-(trifluoromethyl)pyridine (1.62 g, 0.01 mol) in concentrated hydrochloric acid (1.7 mL), was treated with ice (2 g) and cooled to 0° C. Sodium nitrite (0.71 g, 0.01 mol) in water (2 mL) was added slowly, the reaction mixture stirred for 5 minutes at 0° C. then treated slowly with a solution of potassium ethyl xanthate (1.92 g, 0.012 mol) in ethanol-water. The reaction mixture was heated at 50-55° C. for 30 minutes, cooled and diluted with diethyl ether and water. The organic layer was washed with brine, dried (MgSO₄) and evaporated in vacuo. The resulting xanthate was dissolved in ethanol (30 mL) and treated with potassium hydroxide (3 g) and refluxed (90° C.) for 2 h. After cooling and filtering, the filtrate was acidified with citric acid and diluted with diethyl ether. The organic layer was washed with brine, dried (MgSO₄) and evaporated in vacuo. Purification by column chromatography on silica gave the (trifluoromethyl)pyridinethiol as a yellow oil (0.79 g, 44%).

### ¹H NMR (360 MHz, CDCl₃) δ 8.57 (1H, d, J=2.0 Hz), 7.74 (1H, dd, J=8.1, 2.0 Hz), 7.54 (1H, d, J=8.1 Hz), 3.62 (1H, s).

(2) This thiol (0.5 g, 2.8 mmol) was reacted first with 2,5-difluorobenzyl bromide and subsequently with 3-chloroperoxybenzoic acid by the procedure described for Intermediate 1 in WO 02/081435 to gave the pyridyl benzyl sulfone as a white powder (0.82 g, 87% over 2 steps).

### ¹H NMR (400 MHz, CDCl₃) δ 8.93 (1H, d, J=2.1 Hz), 8.18 (1H, dd, J=8.1, 2.1 Hz), 7.80 (1H, d, J=8.1 Hz), 7.21-7.17 (1H, m), 7.10-7.04 (1H, m), 6.93-6.88 (1H, m), 4.46 (2H, s).

(3) This sulfone (50 mg, 0.15 mmol) in tetrahydrofuran (5 mL) at 0° C. was treated with potassium tert-butoxide (17 mg, 0.15 mmol), then with 2,2-bis(2-iodoethyl)-1,3-dioxolane (H. Niwa et al, J. Am. Chem. Soc., 1990, 112, 9001) (86 mg, 0.23 mmol), stirred for 1 h at room temperature and then for 1 h at 70° C. The cooled reaction mixture was treated with more potassium tert-butoxide (1.2 equivalents) and 2,2-bis(2-iodoethyl)-1,3-dioxolane (0.3 equivalents). After heating at 70° C. for 1 h, then cooling to room temperature, the reaction mixture was diluted with diethyl ether and water, the layers separated and the organic layer washed with water and brine, dried (MgSO₄) and evaporated in vacuo. Purification by column chromatography on silica gave the desired cyclohexanone cyclic ketal (38 mg, 56%) as a white solid.
¹H NMR (360 MHz, CDCl₃) δ 8.68 (1H, d, J=2.0 Hz), 7.92 (1H, dd, J=2.0, 8.1 Hz), 7.73 (1H, d, J=8.1 Hz), 7.19-7.07 (2H, m), 6.90-6.82 (1H, m), 3.99-3.88 (4H, m), 2.7 (2H, vbrm), 2.5 (2H, vbrappt), 1.85 (2H, brappd), 1.54-1.26 (2H, m).
(4) This ketal (30 mg, 0.065 mmol) was heated at 50° C. overnight with p-toluenesulfonic acid (15 mg) in 80% acetic acid-water. The reaction mixture was partitioned between diethyl ether and water and the organic layer washed with saturated aqueous sodium hydrogencarbonate solution and brine, dried (MgSO₄) and evaporated in vacuo. Purification by column chromatography on silica gave the cyclohexanone (25 mg, 92%) as a white solid.

### ¹H NMR (400 MHz, CDCl₃) δ 8.67 (1H, d, J=2.0 Hz), 7.97 (1H, dd, J=8.1, 2.0 Hz), 7.77 (1H, d, J=8.1 Hz), 7.28-7.16 (2H, m), 6.99-6.90 (1H, m), 3.01-2.97 (2H, m), 2.68-2.57 (4H, m), 2.26-2.17 (2H, m).

(5) The cyclohexanone was converted to the title amine by the procedure of Intermediate A, except that the borohydride reduction was carried out at −78° C. M/Z 421 (MH⁺).

### Sulfonyl Chlorides

The sulfonyl chlorides used in these examples were typically commercially available, or available by literature routes. Representative syntheses include the following:

2-methyl-1-propanesulfonyl chloride

To 2-methyl-1-propanethiol (200 mg, 2.22 mmol) at 0° C. in acetonitrile under nitrogen was added KNO₃ (561 mg, 5.5 mmol) then sulfuryl chloride (0.45 ml, 5.5 mmol). The reaction mixture was stirred at 0° C. for 3 h, diluted with NaHCO₃ and extracted with ethyl acetate. The combined organic layers were washed with brine, dried (MgSO₄) and evaporated to give the sulfonyl chloride (293 mg, 95%)

2-chlorosulfonyl-1-methylimidazole
Bleach (12% w/w aq, 110 ml) was cautiously added dropwise to a solution of 2-mercapto-1-methylimidazole (2.0 g) in conc. H₂SO₄ (50 ml) cooled to 0° C. After stirring 30 minutes at 0° C. the mixture was diluted with H₂O (30 ml) and dichloromethane (30 ml). The aqueous layer was re-extracted with dichloromethane and the combined organic layers dried (MgSO₄) and evaporated to give the product as an oil (730 mg).
5-chlorosulfonyl-1-methyltetrazole
Cl₂(g) was bubbled through a solution of 5-mercapto-1-methyltetrazole (1.518 g) in 2N HCl (25 ml) at 0° C. After 15 minutes the solid precipitate (880 mg) was filtered off and washed with H₂O.

### Example 1

### methanesulfonic acid, N-[4-(4-chlorobenzenesulfonyl)-4-(2,5-difluorophenyl)-cyclohexyl]-amide

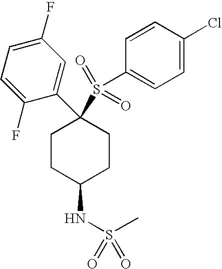

Methanesulfonyl chloride (24 μL, 0.31 mmol) was added to a solution of Intermediate A (100 mg, 0.28 mmol) and triethylamine (77 μL, 0.56 mmol) in dichloromethane (1.5 ml) at 0° C. After stirring at ambient temperature for 12 hours, the reaction was partitioned between water (50 ml) and dichloromethane (50 ml), the phases separated and the aqueous layer washed twice more with dichloromethane. The combined organic layers were washed with 1N HCl. the acidic layer extracted twice with dichloromethane and the combined organics dried over K₂CO₃ and concentrated. Flash column chromatography eluting with 60/40 hexane/ethyl acetate afforded the title compound (88.5 mg).

### MS(ES⁻) [M−H] 462, 464.

The following examples were prepared by the same procedure, using the appropriate sulfonyl or sulfinyl chloride:

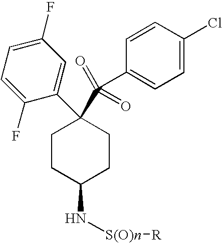

| | | | MS |
|---|---|---|---|
| | | | (ES⁻ (MH⁻) |
| | | | unless otherwise |
| Example | n | R | stated) |
| 2 | 2 | CH₂CF₃ | 530, 532 |
| 3 | 2 | ⁿPr | 490, 492 |
| 4 | 2 | benzyl | 538, 540 |
| 5 | 2 | phenyl | 524, 526 |
| 6 | 2 | 2-thienyl | 530, 532 |
| 7 | 2 | ethyl | 476, 478 |
| 8 | 2 | 5-chloro-2-thienyl | 566, 568 |
| | | | (M + H)⁺ |
| 9 | 2 | n-butyl | 504, 506 |
| 10 | 2 | 2-fluorophenyl | 542, 544 |
| 11 | 2 | 3-fluorophenyl | 542, 544 |
| 12 | 2 | 4-fluorophenyl | 542, 544 |
| 13 | 2 | 2-pyridyl | 525, 527 |
| 14 | 2 | 5-methyl-2-thienyl | 546, 548 |
| | | | (MH)⁺ |
| 15 | 2 | 5-isothiazolyl | 531, 533 |
| 16 | 2 | 4-chloro-2-thienyl | 564, 566, 568 |
| 17 | 2 | 2-(trifluoromethyl)phenyl | 616/618 |
| | | | [M + Na]⁺. |
| 18 | 2 | CH₂CH(CH₃)₂ | 506, 508 |
| | | | [MH]⁺ |
| 19 | 2 | CH₂SO₂Me | 542, 544 |
| | | | [MH]⁺ |
| 20 | 2 | 2-methylphenyl | 540, 542 |
| | | | [MH]⁺ |
| 21 | 2 | 4-Me-1,2,4-triazol-3-yl | 529, 531 |
| 22 | 2 | 2-thiazolyl | 531, 533 |
| 23 | 2 | chloromethyl | 494, 496, 498 |
| 24 | 2 | 2-furyl | 514, 516 |
| 25 | 2 | 2-chlorophenyl | 558, 560 |
| 26 | 2 | 2-cyanophenyl | 549, 551 |
| 27 | 2 | 3,5-di-Me-isoxazol-4-yl | 543, 545 |
| 28 | 2 | 3-thienyl | 530, 532 |
| 29 | 2 | 3-chloropropyl | 524, 526 |
| 30 | 2 | 1-Me-tetrazol-5-yl | 532, 543 |
| | | | [MH]⁺ |
| 31 | 2 | 1,2,4-triazol-3-yl | 517, 519 |
| | | | [MH]⁺ |
| 32 | 2 | 3-chloro-2-thienyl | 564, 566 |
| 33 | 2 | 1-Me-imidazol-5-yl | 530, 532 |
| | | | [MH]⁺ |
| 34 | 2 | 1-Me-imidazol-4-yl | 530, 532 |
| | | | [MH]⁺ |
| 35 | 2 | 1-Me-imidazol-2-yl | 528, 530 |
| 36 | 2 | 2-bromophenyl | 430 [MH-4-Cl-PhSO₂]⁺ |
| 37 | 2 | 3-Cl-1,5-di-Me-pyrazol-3-yl | — |
| 38 | 2 | CH₂CO₂Me | 520, 522 |
| 39 | 1* | Me | 272 [M-ArSO₂⁻]⁺, |
| | | | 448[MH]⁺ |
| | | | 470[M + Na]⁺ |
| 40 | 2 | 3-bromo-2-thienyl | 610, 612 |
| 41 | 2 | 4-bromo-2-thienyl | 610, 612 |
| 42** | 2 | 2-methoxy-5-chlorophenyl | 588, 590 |
| 43** | 2 | 2-methoxyphenyl | 554, 556 |

*-MeSOCl-Corey et al J. Am. Chem. Soc., 90, 5548-52 (968).
**-obtained as a 1:1 mixture, separated by chromatography, using the sulfonyl chlorides obtained from treating 2-methoxybenzenethiol with sulfuryl chloride.

### Example 44

### [4-(4-Chlorobenzenesulfonyl)-4-(2,5-difluorophenyl)cyclohexyl]aminosulfonyl-acetamide

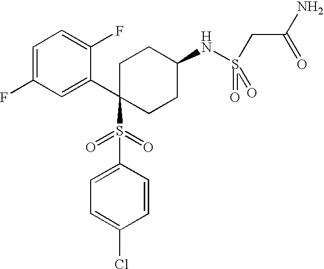

Prepared from the ester of Example 38 by treatment with NH₃ (25% aqueous solution) in ethanol. m/z(ES−)=505/507.

### Example 45

### 2-hydroxyethanesulfonic acid, N-[4-(4-chlorobenzenesulfonyl)-4-(2,5-difluorophenyl)-cyclohexyl]-amide

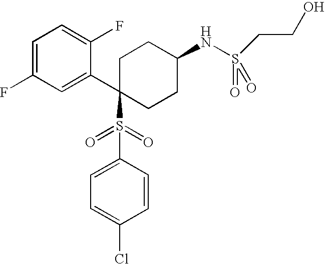

Prepared from the ester of Example 38 by reduction with LiAIH₄ in tetrahydrofuran. m/z(ES−)=492/494.

### Example 46

### cyanomethanesulfonic acid, N-[4-(4-chlorobenzenesulfonyl)-4-(2,5-difluorophenyl)-cyclohexyl]-amide

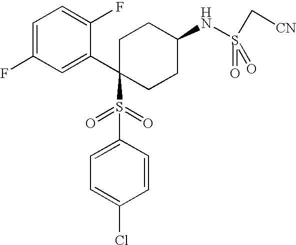

Prepared from the amide of Example 44 by treatment with thionyl chloride and a catalytic amount of dimethylformamide in toluene.
m/z(ES−)=487/489

### Example 47

### trifluoromethanesulfonic acid, N-[4-(4-chlorobenzenesulfonyl)-4-(2,5-difluorophenyl)-cyclohexyl]-amide

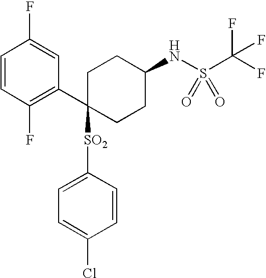

Intermediate A (110 mg, 0.29 mmol) in dichloromethane (3 ml) cooled to 0° C. was treated with triethylamine (99 μL, 0.43 mmol) followed by triflic anhydride (117 μL, 0.71 mmol). The reaction was stirred at 0° C. for 2.5 hours, slowly warming to ambient temperature, then diluted with ethyl acetate, washed with 2N sodium hydroxide, dried (MgSO₄) and evaporated to an orange oil which was purified by chromatography 15% ethyl acetate / hexane to yield a white solid (16 mg). ¹H NMR (360MHz, CDCl₃) δ 7.39-7.30 (4H, m), 7.09-7.04 (2H, m) 6.88-6.81 (1H, m), 5.86-5.84 (1H, m), 3.82-3.80 (1H, m), 2.64-2.42 (4H, m), 2.07-2.02 (2H, m), 1.66-1.59 (2H, m).
m/z=540, 542[M+Na]⁺

### Example 48

### methanesulfonic acid, N-[4-(4-chlorobenzenesulfonyl)-4-(2,5-difluorophenyl)-cyclohexyl]-N-methyl-amide

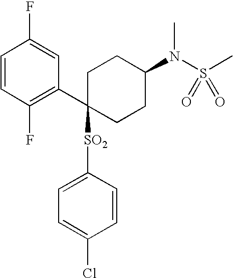

Prepared from the product of Example 1 by treatment with NaH and Mel in tetrahydrofuran. m/z=500, 502 [MNa]⁺

### Examples 49-61, 68

The following examples were prepared similarly to Example 1, substituting Intermediate B for Intermediate A and using the appropriate sulfonyl chloride:

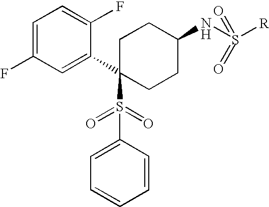

| | | MS |
|---|---|---|
| | | (MH⁺) unless |
| Example | R | otherwise stated |
| 49 | Me | 499 |
| 50 | 2-furyl | [MNa⁺] 572 |
| 51 | ethyl | [MH⁻] 513 |
| 52 | CH₂SO₂Me | [MNa⁺] 598 |
| 53 | 1-Me-imidazol-4-yl | 564 |
| 54 | 5-isothiazolyl | 567 |
| 55 | 2-pyridyl | [MNa⁺] |
| | | 583, 561 |
| 56 | 5-chloro-2-thienyl | 600, 602 |
| 57 | n-propyl | 526 |
| 58 | 2-thienyl | 566. |
| 59 | 6-chloro-3-pyridyl | 596, 598 |
| 60 | 3-thienyl | 566 |
| 61* | vinyl | 510, [MH-SO₂Ar⁺] |
| | | 300 |
| 68 | 2-MeCO₂-3-thienyl | — |

*-prepared using 2-chloroethanesulfonyl chloride

### Example 62

### trifluoromethanesulfonic acid, N-[4-(2,5-difluorophenyl)-4-(4-trifluoromethyl-benzenesulfonyl)-cyclohexyl]-amide

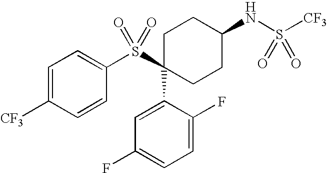

Intermediate B (170 mg, 0.41 mmol) in dry dichloromethane (5 ml) under nitrogen was treated at 0° C. with triethylamine (80 μl, 0.62 mmol) and triflic anhydride (133 μl, 0.82 mmol). The reaction was allowed to warm to room temperature, stirred for 3 h. diluted with dichloromethane, washed with water, brine, dried (MgSO₄) filtered and evaporated. The residue was purified by flash chromatography eluting with iso-hexane/ethyl acetate (1:1) to give a white solid (60 mg).
¹H NMR δ (ppm) (DMSO): 1.46-1.53 (2H, m), 1.81 (1H, s), 1.84 (1H, s), 2.41 (2H, t, J=13.1 Hz), 2.56-2.59 (1H, m), 2.59 (1H, d, J=2.7 Hz), 3.64 (1H, s), 7.10-7.23 (2H, m), 7.30-7.36 (1H, m), 7.60 (2H, d, J=8.2 Hz), 7.94 (2H, d, J=7.6 Hz), 9.77 (1H, d, J=7.6 Hz).

### Example 63

### 2-(morpholin-4-yl)ethanesulfonic acid, N-[4-(2,5-difluorophenyl)-4-(4-trifluoromethyl-benzenesulfonyl)-cyclohexyl]-amide

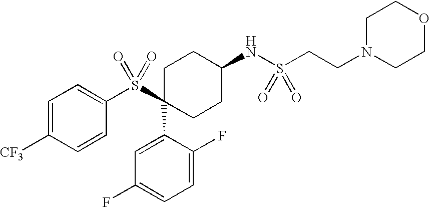

Prepared from Example 61 by reaction with excess morpholine in dry dimethylformamide. MS [MH⁺] 597

### Example 64

### 6-(morpholin-4-yl)pyridine-3-sulfonic acid, N-[4-(2,5-difluorophenyl)-4-(4-trifluoromethyl-benzenesulfonyl)-cyclohexyl]-amide

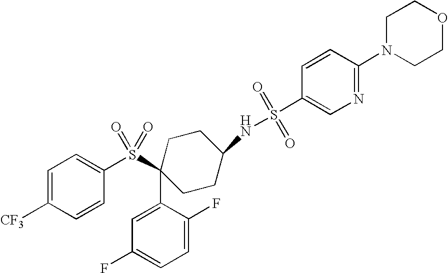

Prepared from example 59 by refluxing in ethanol with morpholine.

### Example 65

### 1,1-dimethylethanesulfinic acid, N-[4-(2,5-difluorophenyl)-4-(4-trifluoromethyl-benzenesulfonyl)-cyclohexyl]-amide

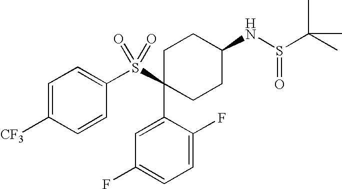

Intermediate B (0.73 g, 1.75 mmol) and 1,1-dimethylethyl sulfinamide (0.21 g, 1.75 mmol) in tetrahydrofuran (20 ml) were treated with titanium (IV) ethoxide (0.36 ml, 1.75 mmol) and heated to 80° C. for 18 hours. The reaction was quenched with water (0.35 ml) and stirred for 10 minutes before filtering through Celite™. The sulfinimine was cooled to −30° C. and treated with L-Selectride™ (1.75 ml, 1.0 mmol solution), and stirred for 2 hours whilst warming to −5° C. The reaction was then quenched with methanol (2 ml), and partitioned between ethyl acetate (50 ml) and brine (50 ml), dried (MgSO₄) and evaporated to dryness. The product was purified by silica gel chromatography eluting with ethyl acetate/hexane mixtures. Yield 120 mg. MS MH=524.

### Example 66

### 1,1-dimethylethanesulfonic acid, N-[4-(2,5-difluorophenyl)-4-(4-trifluoromethyl-benzenesulfonyl)-cyclohexyl]-amide

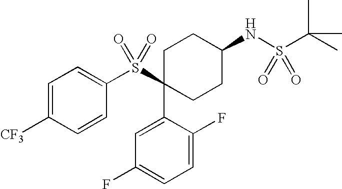

Prepared from the product from Example 65 by oxidation with m-chloroperoxybenzoic acid in dichloromethane. MS MH=540.

### Example 67

### 2-(pyrrolidin-1-yl)ethanesulfonic acid, N-[4-(2,5-difluorophenyl)-4-(4-trifluoromethyl-benzenesulfonyl)-cyclohexyl]-amide

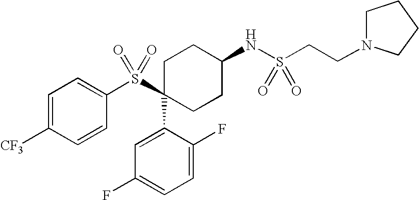

Prepared as in Example 63, substituting pyrrolidine for morpholine.

### MS [MH⁺] 581

### Examples 69-73

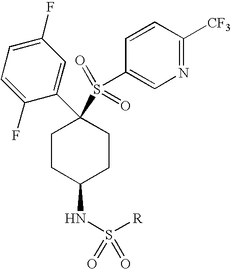

The following sulfonamides were prepared by the procedure of Example 1 using Intermediate C and the appropriate sulfonyl chloride.

| Example | R | MS (MH⁺) |
|---|---|---|
| 69 | methyl | 499 |
| 70 | 2-thienyl | 567 |
| 71 | 5-isothiazolyl | 568 |
| 72 | n-propyl | 527 |
| 73* | 2-chloro-2-propyl | 561, 563 |

*Isopropylsulfonyl chloride was used.

### Example 74

### trifluoromethanesulfonic acid, N-[4-(2,5-difluorophenyl)-4-(6-trifluoromethyl-pyridine-3-sulfonyl)-cyclohexyl]-amide

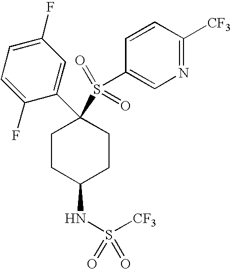

Intermediate C (100 mg) in dichloromethane (5 ml) and treated with triethylamine (1 equivalent) and cooled to −78 ° C. Trifluoromethanesulfonic anhydride (2 equivalents) was added, the reaction mixture warmed to −40 ° C. and stirred at this temperature for 3 h. The mixture was quenched with aqueous citric acid, diluted with ethyl acetate and warmed to room temperature. The organic phase was separated, washed with brine, dried (MgSO₄), filtered and evaporated in vacuo. Purification by column chromatography (eluting with 5/1 hexane/ethyl acetate) gave the title compound (120 mg, 91%) as a white powder. ¹H NMR (CDCl₃, 400 MHz) 8.60 (1H, d, J=1.9), 7.91 (1H, dd, J=8.2, 1.9), 7.74 (1H, d, J=8.2), 7.26-7.10 (2H, m), 6.88-6.81 (1H, m), 5.70 (1H, brd, J=5), 3.83 (1H, brs), 2.64-2.48 (4H, m), 2.11-2.07 (2H, m), 1.70-1.65 (2H, m).

### Example 75

### trifluoromethanesulfonic acid, N-[4-(5-bromo-2-fluorophenyl)-4-(6-trifluoromethyl-pyridine-3-sulfonyl)-cyclohexyl]-amide

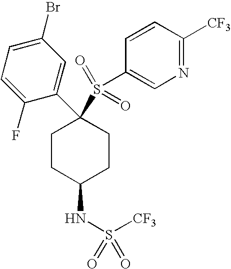

Prepared by the procedures of Intermediate C (using 2-fluoro-5-bromobenzyl bromide in Step 2) and Example 1 (using trifluoromethanesulfonyl chloride). M/Z=613, 615 (MH⁺).

### Example 76

### trifluoromethanesulfonic acid, N-[4-(5-cyano-2-fluorophenyl)-4-(6-trifluoromethyl-pyridine-3-sulfonyl)-cyclohexyl]-amide

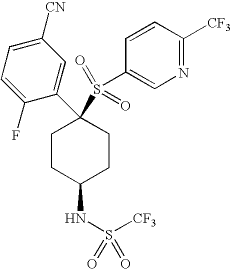

Prepared from Example 75 (45 mg) by heating with copper cyanide (4 equivalents), pyridine (1 drop) in dimethylformamide at 180° C. overnight. M/Z=559 (MH⁺).

### Example 77

### trifluoromethanesulfonic acid, N-[4-(2-fluoro-5-methyl-phenyl)-4-(6-trifluoromethyl-pyridine-3-sulfonyl)-cyclohexyl]-amide

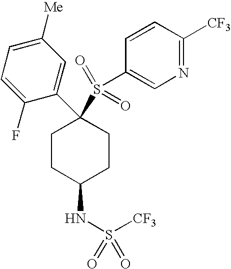

Prepared from Example 75 by treatment with cesium fluoride (2.2 equivalents), tri-tert-butylphosphine (12 mol %), tetramethyltin (2 equivalents), and Pd₂(dba)₃ (3 mol %) at 100° C. in dioxan for 3 h M/Z=549 (MH⁺).

### Example 78

### trifluoromethanesulfonic acid, N-[4-(2-fluoro-5-(hydroxymethyl)-phenyl)-4-(6-trifluoromethyl-pyridine-3-sulfonyl)-cyclohexyl]-amide

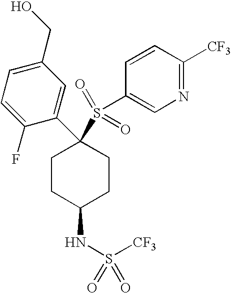

Prepared from Example 75 by (i) treatment with CsF (2.2 equivalents), tri-tert-butylphosphine (12 mol %), tributylvinyltin (2 equivalents), and Pd₂(dba)₃ (3 mol %) in dioxan at 100° C. for 2 h.; (ii) treatment of the resulting styrene with ozone at −78° C. in dichloromethane/methanol; and (iii) reduction of the resulting aldehyde at −78° C. with sodium borohydride (2 equivalents) in ethanol. M/Z=547 (M−OH+H⁺).

### Example 79

### trifluoromethanesulfonic acid, N-[4-(2,5-difluorophenyl)-4-(4-vinylbenzenesulfonyl)-cyclohexyl]-amide

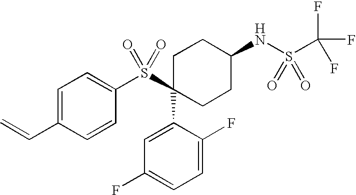

Prepared from Example 47 by treatment with tri-t-butylphosphine, cesium fluoride, Pd₂(dba)₃ and tributyl(vinyl)tin in dioxan at 100° C. for 2 hours.

### Example 80

### trifluoromethanesulfonic acid, N-[4-(2,5-difluorophenyl)-4-(4-formylbenzenesulfonyl)-cyclohexyl]-amide

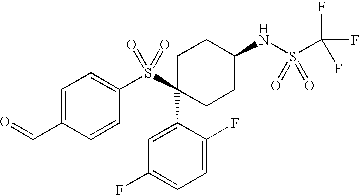

Prepared from Example 79 by treatment with ozone in dichloromethane/methanol at −78° C.

### Example 81

### 4-{1-(2,5-difluoroephenyl)-4-[(trifluoromethanesulfonel)amino]-cyclohexanesulfonyl}-benzaldehyde oxime

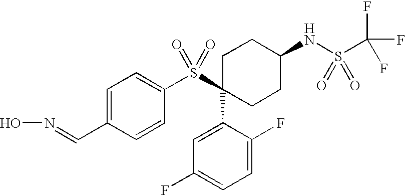

Prepared from Example 80 by treatment with hydroxylamine hydrochloride and sodium acetate in refluxing ethanol.

### Example 82

### trifluoromethanesulfonic acid, N-[4-(2,5-difluorophenyl)-4-(4-(fluoromethyl)benzenesulfonyl)-cyclohexyl]-amide

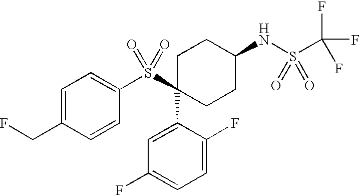

Prepared from Example 80 by (i) reduction with sodium borohydride in dry tetrahydrofuran at 0° C.; and (ii) treatment of the resulting benzyl alcohol with diethylaminosulfur trifluoride in dry dichloromethane at −78° C.

### Example 83

### trifluoromethanesulfonic acid, N-[4-(2,5-difluorophenyl)-4-(4-(difluoromethyl)benzenesulfonyl)-cyclohexyl]-amide

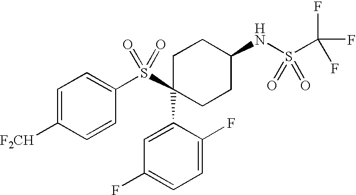

Prepared from Example 80 by treatment with diethylaminosulfur trifluoride in dry dichloromethane at room temperature.

### Example 84

### trifluoromethanesulfonic acid, N-[4-(4-cyanobenzenesulfonyl)-4-(2,5-difluorophenyl)-cyclohexyl]-amide

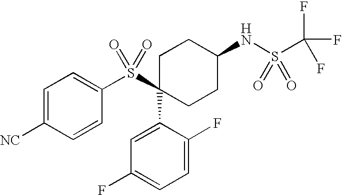

Prepared from Example 81 by treatment with triphenyl phosphine and carbon tetrachloride in acetonitrile.

### Example 85

### trifluoromethanesulfonic acid, N-[4-(benzenesulfonyl)-4-(2,5-difluorophenyl)-cyclohexyl]-amide

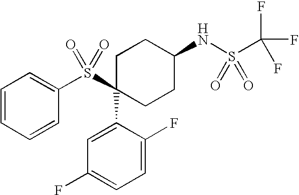

Prepared from Example 47 by hydrogenation over 10% palladium on carbon.

1. A method of treating cancer in a mammal in need of such treatment comprising administering to said mammal a therapeutically effective amount of a compound of formula I: 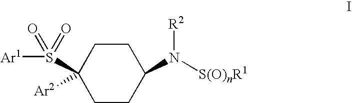
wherein n is 1 or 2;
R¹ represents CF₃ or C₁₋₆alkyl, C₂₋₆alkenyl, C₃₋₉cycloalkyl or C₃₋₆cycloalkylC₁₋₆alkyl, any of which may bear up to 2 substituents selected from halogen, CN, CF₃, OR³, COR³, CO₂R³, OCOR⁴, SO₂R⁴, N(R⁵)₂, and CON(R⁵)₂,
or R¹ represents aryl, arylC₁₋₆alkyl, C-heterocyclyl or C-heterocyclylC₁₋₆alkyl;
R² represents H or C₁₋₄alkyl;
R³ represents H, C₁₋₄alkyl, phenyl or heteroaryl;
R⁴ represents C₁₋₄alkyl, phenyl or heteroaryl;
R⁵ represents H or C₁₋₄alkyl, or two R⁵ groups together with a nitrogen atom to which they are mutually attached complete an azetidine, pyrrolidine, piperidine, morpholine, thiomorpholine or thiomorpholine-1,1-dioxide ring;
Ar¹ and Ar² independently represent phenyl or heteroaryl, either of which bears 0-3 substituents independently selected from halogen, CN, NO₂, CF₃, CHF₂, OH, OCF₃, CHO, CH═NOH, C₁₋₄alkoxy, C₁₋₄alkoxycarbonyl, C₂₋₆acyl, C₂₋₆alkenyl and C₁₋₄alkyl which optionally bears a substituent selected from halogen, CN, NO₂, CF₃, OH and C₁₋₄alkoxy;
“aryl” at every occurrence thereof refers to phenyl or heteroaryl which optionally bear up to 3 substituents selected from halogen, CN, NO₂, CF₃, OCF₃, OR³, COR³, CO₂R³, OCOR⁴, N(R⁵)₂, CON(R⁵)₂ and optionally-substituted C₁₋₆alkyl, C₁₋₆alkoxy, C₂₋₆alkenyl or C₂₋₆alkenyloxy wherein the substituent is selected from halogen, CN, CF₃, phenyl, OR³, CO₂R³, OCOR⁴, N(R⁵)₂ and CON(R⁵)₂; and
“C-heterocyclyl” and “N-heterocyclyl” at every occurrence thereof refer respectively to a heterocyclic ring system bonded through carbon or nitrogen, said ring system being non-aromatic and comprising up to 10 atoms, at least one of which is O, N or S, and optionally bearing up to 3 substituents selected from oxo, halogen, CN, NO₂, CF₃, OCF₃, OR³, COR³, CO₂R³, OCOR⁴, OSO₂R⁴, N(R⁵)₂, CON(R⁵)₂ and optionally-substituted phenyl, C₁₋₆alkyl, C₁₋₆alkoxy, C₂₋₆alkenyl or C₂₋₆alkenyloxy wherein the substituent is selected from halogen, CN, CF₃, OR³, CO₂R³, OCOR⁴, N(R⁵)₂ and CON(R⁵)₂;
or a pharmaceutically acceptable salt thereof.

2. The method Use according to claim 1 wherein n is 2.

3. The method Use according to claim 1 wherein Ar¹ is 6-trifluoromethyl-3-pyridyl, 4-chlorophenyl or 4-trifluoromethylphenyl and Ar² is 2,5-difluorophenyl.

4. The method compound according to claim 1 wherein said compound is a compound of formula II: 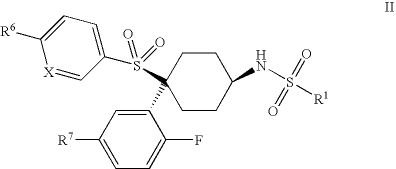
wherein X represents N or CH;
R⁶ represents H, F, Cl, Br, CN, CF₃, CH═CH₂ or CH₃;
R⁷ represents F, Cl, Br, CN, CH₃ or CH₂OH; and
R¹ is as defined in claim 1;
or a pharmaceutically acceptable salt thereof.

5. The method according to claim 4 wherein R¹ is CF₃.

6. The method according to claim 4 wherein said compound is trifluoromethanesulfonic acid, N-[4-(2,5-difluorophenyl)-4-(6-trifluoromethyl-pyridine-3-sulfonyl)-cyclohexyl]-amide or a pharmaceutically acceptable salt thereof.

7. The method according to claim 1 wherein the cancer is selected from breast, prostate, colon, ovarian, colorectal and lung cancers.

8. The method according to claim 1 wherein the cancer is lymphoma or leukemia.

9. The method according to claim 8 wherein the cancer is T-ALL.

10. The method according to claim 1 wherein the compound of formula I is administered in combination with another anti-cancer agent or therapeutic agent, optionally in conjunction with radiation therapy.

11. The method according to claim 10 wherein said other anti-cancer agent or therapeutic agent is selected from the group consisting of: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, a γ-secretase and/or NOTCH inhibitor, an agent that interferes with receptor tyrosine kinases (RTKs), and an agent that interferes with a cell cycle checkpoint.

12. (canceled)
